(19) United States
(12) Patent Application Publication
(10) Pub. No.: US 2009/0258949 A1
(43) Pub. Date: Oct. 15, 2009

(54) AMORPHOUS FORM OF CINACALCET

(76) Inventors: Johannes Ludescher, Breitenbach (AT); Ulrich Griesser, Axams (AT); Doris Braun, Dornbirn (AT); Josef Wieser, Polling (AT)

Correspondence Address: MANELLI DENISON & SELTER, 2000 M STREET NW SUITE 700, WASHINGTON, DC, 20036-3307, US

(21) Appl. No.: 12/303,438

(22) PCT Filed: Jun. 25, 2007
(86) PCT No.: PCT/EP07/05600

§ 371 Date: Dec. 4, 2008

(30) Foreign Application Priority Data
Jun. 27, 2006 (EP) 06116134.5
Oct. 19, 2006 (EP) 06122584.3

(51) Int. Cl. A61K 31/135; A61P 5/00

(52) U.S. Cl. 514/657

(57) ABSTRACT

The present invention relates to dispersions of stable amorphous cinacalcet hydrochloride in a matrix material, methods for the preparation thereof, and pharmaceutical compositions comprising said dispersions.

The present invention relates to dispersions of stable amorphous cinacalcet hydrochloride in a matrix material, methods for the preparation thereof, and pharmaceutical compositions comprising said dispersions.

Cinacalcet hydrochloride, N-[1-(R)-(−)-(1-naphthyl)ethyl]-3-[3-(trifluoromethyl)phenyl]-1-aminopropane hydrochloride, shown as Compound (I) below

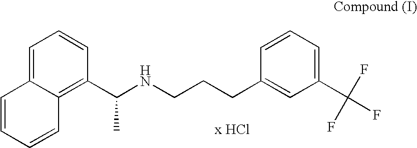

is a novel second generation calcimimetic that modulates the extra cellular calcium sensing receptor (CaR) by making it more sensitive to the calcium suppressive effects on parathyroid hormone (PTH). It is used in a treatment for primary and secondary hyperparathyroidism. Hyperparathyroidism is characterized by high levels of circulating calcium due to an increased secretion of parathyroid hormone by one or more of the parathyroid glands. Hyperparathyroidism can lead to e.g. osteoporosis; patients with renal failure suffering from secondary hyperparathyroidism have for example an increased risk of renal bone disease, soft-tissue calcifications and vascular disease.

The preparation of Cinacalcet is, for example, described in Drugs of the Future 2002, 27(9), 831-836 and its use in the treatment of primary and secondary hyperparathyroidism has been the subject of several research articles, e.g. Expert opinion on investigational drugs (2003), 12(8), 1413-21.

Cinacalcet is sold e.g. in US as Sensipar® in the form of tablets. Sensipar® is to be used in the treatment of hyperparathyroidism and of hypercalcemia.

The isolation of Cinacalcet as hydrochloride is not described in the patent literature. U.S. Pat. No. 6,211,244 exemplifies the synthesis and isolation of analogues. Hydrochlorides of these analogues are prepared by the precipitation using gaseous HCl in ether or hexane in combination with gaseous HCl in ether. This method is not applicable to large scale synthesis.

Amorphous products often show improved absorption in humans. As a result, the amorphous form may show an increasing bioavailability.

However, amorphous products often show a chemical stability which renders them unsuitable for the preparation of medicaments and amorphous products as such are sometimes too hygroscopic to be suitable for the preparation of pharmaceutical formulations.

There is thus a need for an amorphous form of cinacalcet hydrochloride which is suitable for application in pharmaceutical compositions.

### SUMMARY OF THE INVENTION

The present invention provides dispersions of stable amorphous cinacalcet hydrochloride in a matrix material, methods for the preparation thereof, and pharmaceutical compositions comprising said dispersions.

### DETAILED DESCRIPTION OF THE INVENTION

The present inventors have identified an amorphous form of Cinacalcet Hydrochloride which is chemically stable upon storage when present as a dispersion in a matrix material. In that form, also the problem of hygroscopicity of amorphous Cinacalcet Hydrochloride is solved, thereby enabling the use of amorphous Cinacalcet Hydrochloride for the preparation of pharmaceutical compositions.

The invention therefore relates to a dispersions of stable amorphous cinacalcet hydrochloride in a matrix material.

By “stable” it is meant that the amorphous form of Cinacalcet Hydrochloride of the invention shows very little degradation upon storage under stress conditions, i.e. there is essentially no decrease in assay of Cinacalcet as measured by HPLC, the measurement being detailed in example 1, the decrease being less than 0.3 area % when stored at 60° C. for 24 hours. In the context of the inventive dispersions, the amorphous form of Cinacalcet Hydrochloride preferably exhibits an increase in impurity levels as measured by HPLC as described above of less than 0.2 area % when stored at 25° C./60% relative humidity for one month, in particular even after storage for 6 months.

“Matrix material” relates to the matrix of a pharmaceutical formulation which is formed by a pharmaceutically acceptable carrier after removal of the solvent in the process for the preparation of a dispersions of stable amorphous cinacalcet hydrochloride in a matrix material described below.

As pharmaceutically acceptable carriers any material described in Encyclopedia of Pharmaceutical Technology (Vol 3, Table Ion page 345) may be used and preferred carriers are macrogels, succinic acid, urea, pectin, desoxycholic acid, galactomannan, urethane, methylcellulose, hydroxypropylcellulose, polyethylenglycol, poloxamers, polyacrylates, polymethylacrylates, hydroxyalkylxanthine, dextrose, sucrose, polyvinylpyrrolidon, galactose, maltose, xylitol, cyclodextrin, mannitol, sorbitol, and in particular polyethylenglycol, e.g. PEG 6000, maltose, sucrose, HPMC (hydroxypropyl-methylcellulose) or HPMCP (hydroxypropyl methylcellulose phthalate).

The invention further relates to processes for the production of a dispersion of stable amorphous cinacalcet hydrochloride in a matrix material of the invention starting from crystalline Cinacalcet Hydrochloride or solutions originating from the synthesis or purification of Cinacalcet Hydrochloride.

Cinacalcet free base may be prepared by methods know in the literature, e.g. by reductive amination of 3-[3-(trifluoromethyl)phenyl]-propionadehyde with 1(R)-(1-naphthyl)ethylamine as disclosed e.g. in Drugs of the future 2002, 27(99), 831-836. Alternatively Cinacalcet hydrochloride or a salt of Cinacalcet with an organic acid or inorganic acid may be used as starting material. A solution of these salts may be used directly as starting material for hydrochloride formation described below or these salts may be converted to the free base, e.g. by means of neutralization of a solution of these salts with a suitable base.

The solution of Cinacalcet hydrochloride may then be provided by mixing of Cinacalcet free base with a hydrochloride source, e.g. aqueous or gaseous HCl, e.g. in stoichiometric amounts or using an excess of the hydrochloride source, e.g. up to 5 equivalents of the hydrochloride source in a solvent or solvent mixture as described above. A preferred way to generate Cinacalcet hydrochloride is the use of a trialkylsilylchloride in combination with a protic solvent as hydrochloride source as described in detail in Co-pending European application EP06116134, herein incorporated by reference.

A very preferred process for the preparation of a solution of Cinacalcet hydrochloride comprises the steps of:

(a) dissolving the free base of Cinacalcet in a protic solvent, and
(b) adding a trialkylsilylchloride, preferably trimethylchlorosilane, in an amount of about one mole equivalent calculated based on Cinacalcet free base. The resulting solution, for example in ethanole, may be used as starting material for the below described processes for the preparation of stable amorphous Cinacalcet hydrochloride.

In one embodiment the process for the production of a stable amorphous form of Cinacalcet hydrochloride comprising the step of removing the solvent from a solution of Cinacalcet hydrochloride in an organic solvent or a mixture of organic solvents.

Removal of the solvent may be effected by spray drying, lyophilization or distillation. Distillation preferably is performed in vacuo.

Preferred solvents include acetone, dichloromethane, dioxane, mixtures of dioxane with water or diethylether, dimethylsulfoxyde, ethylacetate, ethylmethylketone, tetrahydrofurane, methanole, ethanole, 1-propanole, 2-propanole, 2-propanole in combination with heptane, water or diethylether, or formic acid.

Alternatively, it is preferred that the solvent or solvent mixture is selected from a ketone, ether, ester, halogenated hydrocarbon, alcohole, hydrocarbon, water, or dimethylsulfoxyde.

A preferred ketone is a C₃-C₈ ketone. A preferred ester is selected from a C₁-C₄ carboxylic acid C₁-C₄ alkylester. A preferred ether is selected from a C₂-C₆ dialkylether, tetrahydrofurane or dioxane. A preferred halogenated hydrocarbon is dichloromethane. A preferred alcohole is a C₁-C₄ alcohole. A preferred hydrocarbon is a C₅-C₈ hydrocarbon.

In the inventive process for the preparation of a dispersion of stable amorphous cinacalcet hydrochloride in a matrix material, a pharmaceutically acceptable carrier is present in the removal step. As pharmaceutically acceptable carriers any material described in Encyclopedia of Pharmaceutical Technology (Vol 3, Table Ion page 345) may be used and preferred carriers are macgrogels, succinic acid, urea, pectin, desoxycholic acid, galactomannan, urethane, methylcellulose, hydroxypropylcellulose, hydroxypropylmethylcellulosephthalate, polyethylenglycol, poloxamers, polyacrylates, polymethylacrylates, hydroxyalkylxanthine, dextrose, sucrose, polyvinylpyrrolidon, galactose, maltose, xylitol, cyclodextrin, mannitol, sorbitol, and in particular polyethylenglycol, e.g. PEG 6000, maltose, sucrose, HPMC (hydroxypropyl-methylcellulose) or HPMCP (hydroxypropyl methylcellulose phthalate).

The invention also relates to a preferred process for the preparation of a dispersion of stable amorphous cinacalcet hydrochloride in a matrix material which process comprises the steps of a) dissolving Cinacalcet free base in a solvent selected from the list consisting of acetone, dichloromethane, dioxane, mixtures of dioxane with water or diethylether, dimethylsulfoxyde, ethylacetate, ethylmethylketone, tetrahydrofurane, methanole, 1-propanole, 2-propanole, and 2-propanole in combination with heptane, water or diethylether to obtain a solution of Cinacalcet and b) adding a hydrochloride source to the Cinacalcet solution obtained from step a), for example aqueous or gaseous HCl, in an amount sufficient to form a solution of Cinacalcet Hydrochloride, for example an amount of the hydrochloride source generating an about stoichiometric amount of hydrochloric acid or an excess of hydrochloric acid, e.g. up to 5 equivalents of hydrochloric acid, c) adding a pharmaceutical acceptable carrier at any stage before step d), and d) removing the solvent by spray drying, lyophilization or distillation. Preferred carriers are as described above. Preferred solvents are also as described above.

The present invention further relates to a process for preparing Cinacalcet hydrochloride, which process preferably comprises

a) dissolving the free base of Cinacalcet in an aprotic solvent,
b) adding at least one equivalent of a protic solvent, for example acetic acid or an alcohol like methanol or n-butanol,
c) treating the solution with at least one equivalent of Trimethylchlorosilane.

In the above process, Cinacalcet can be dissolved for example in an aprotic solvent like acetonitrile or ethyl acetate.

The present invention also relates to pharmaceutical compositions comprising a dispersion of stable amorphous cinacalcet hydrochloride in a matrix material.

Preferred pharmaceutical compositions of the invention are oral dosage forms such as tablets, capsules, powders for oral suspension, pills and granules. For example the dispersion of stable amorphous cinacalcet hydrochloride in a matrix material of the invention can be formulated as tablets for oral administration comprising from 20 mg to 300 mg and in particular from 30 mg to 120 mg Cinacalcet Hydrochloride, and further comprising pre-gelatinized starch, microcrystalline cellulose, povidone, crospovidone, colloidal silicon dioxide and magnesium stearate, preferably in amounts equivalent to the marketed product Sensipar® as sold in the US on the priority date. Preferably the tablets are also coated with color, clear film coat and/or carnauba wax.

The invention further relates to a method of treating primary and secondary hyperparathyroidism in a mammal comprising using a dispersion of stable amorphous cinacalcet hydrochloride in a matrix material. The invention further relates to the use of a dispersion of stable amorphous cinacalcet hydrochloride in a matrix material in the preparation of a medicament for the treatment of hyperparathyroidism, in particular for the prevention of treatment of osteoporosis, increased risked of renal bone disease, soft-tissue calcifications and vascular disease associated with hyperparathyroidism.

The stable amorphous form of Cinacalcet Hydrochloride as obtained according to example 1 was analyzed by X-ray powder diffraction diagrams. The X-ray diffraction pattern was obtained using a Siemens D-5000 diffractometer (Bruker AXS, Karlsruhe, D) equipped with a theta/theta goniometer, a CuKα radiation source, a Goebel mirror (Bruker AXS, Karlsruhe, D), a 0.15° soller slit collimator and a scintillation counter. The patterns were recorded at a tube voltage of 40 kV and a tube current of 35 mA, applying a scan rate of 0.005° 2θs−1 in the angular range of 2 to 40° 2θ.

### DESCRIPTION OF THE FIGURE

FIG. 1: PXRD of amorphous Cinacalcet×HCl according to example 1

### EXAMPLES

The following examples describe the present invention in detail, but they are not to be construed to be in any way limiting for the present invention.

### Example 1

50.2 mg Cinacalcet hydrochloride was dissolved in 2 ml of acetone at room temperature. After evaporating the solvent from a watch glass the amorphous form was obtained.

Yield: 50 mg.

In an analogy to example 1 amorphous Cinacalcet hydrochloride was prepared using the solvent or solvent mixtures described in table 1

TABLE 1
Preparation of amorphous Cinacalcet

| Solvent | Second solvent | Form |
|---|---|---|
| Acetone | | amorphous |
| Dichloromethane | | amorphous |
| Dioxane | | amorphous |
| | +water | Amorphous |
| DMF | | Amorphous |
| DMSO | | Amorphous |
| Ethylacetate | | Amorphous |
| THF | | Amorphous |
| Methanol | | Amorphous |
| 1-Propanol | | Amorphous |
| 2-Propanol | | Amorphous |
| | +heptanes | Amorphous |
| | +water | Amorphous |
| | +ether | Amorphous |

### Example 2

A sample of amorphous Cinacalcet Hydrochloride from Example 1 was stressed in a closed vial at 60° C. for 24 hours. No decrease in assay was observed when measured by HPLC under the following conditions:

column: YMC-Pro C18 5 μm, 150×4.6 mm; eluent: sulfamic acid/water, mobile phase A: 7.768 g sulfamic acid in 2000 g of water; mobile phase B: 7.768 g sulfamic acid in 500 g of water; measurement at a wavelength of 254 nm; inj. Vol 7 μm; temperature 40° C.
sample preparation: approximately 10 mg of sample dissolved in 25 ml of eluent B gradient:

| t(min) | % A | % B |
|---|---|---|
| 0 | 70 | 30 |
| 10 | 40 | 60 |
| 14 | 40 | 60 |
| 15 | 0 | 100 |
| 17 | 0 | 100 |
| 18 | 70 | 30 |

Area %/mg of starting material and stressed sample were compared

### Example 3

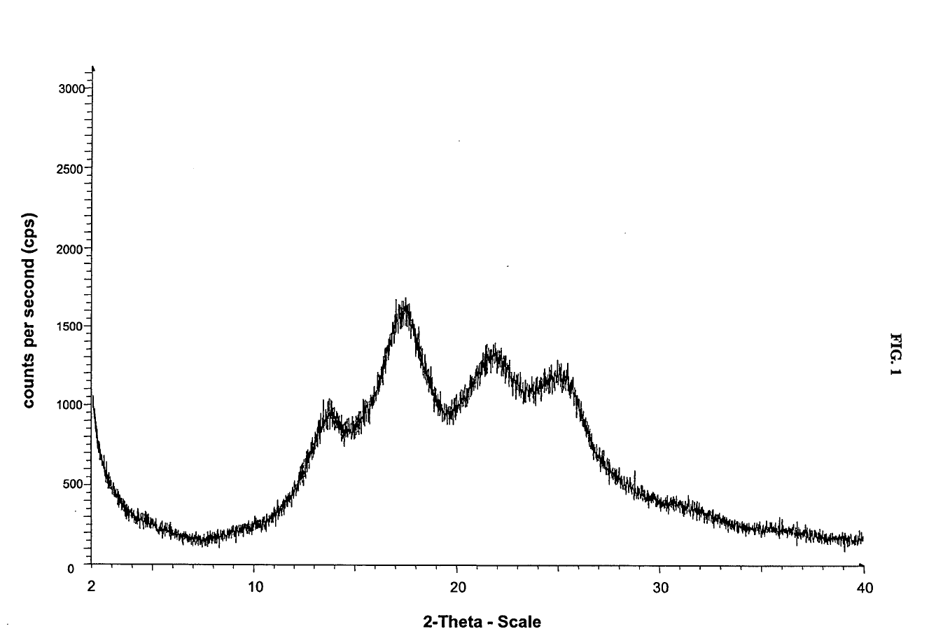

Stable amorphous Cinacalcet Hydrochloride from Example 1 was analyzed by XRPD. The obtained spectrum is shown in FIG. 1.

1. Dispersion of stable amorphous cinacalcet hydrochloride in a matrix material.

2. Process for the preparation of a dispersion of stable amorphous cinacalcet hydrochloride in a matrix material comprising:
a) dissolving Cinacalcet free base in an organic solvent or mixture of organic solvents to obtain a solution of Cinacalcetl and
b) adding a hydrochloride source to the Cinacalcet solution obtained from step a), in an amount sufficient to form a solution of Cinacalcet Hydrochloride;
c) adding a pharmaceutical acceptable carrier at any stage before step d); and
d) removing the solvent to provide a dispersion of stable amorphous Cinacalcet hydrochloride in a matrix material comprising the carrier.

3. Process according to claim 2, wherein the organic solvent solvent is comprises at least one selected from the group consisting of acetone, dichloromethane, dioxane, mixtures of dioxane with water or diethylether, dimethylsulfoxyde, ethylacetate, ethylmethylketone, tetrahydrofurane, methanole, 1-proanole, 2-propanole, and 2-propanole in combination with heptane, water or diethylether, or formic acid.

4. Pharmaceutical composition comprising a dispersion of stable amorphous cinacalcet hydrochloride in a matrix material and a suitable excipient.

5. A method of treating primary and secondary hyperparathyroidism in a mammal comprising feeding a dispersion of stable amorphous Cinacalcet hydrochloride in a matrix material to a mammal.

6. Process according to claim 2, wherein the carrier is added after step b).

7. Process according to claim 2, wherein the solvent is removed by spray drying, lyophilization or distillation.

8. Process according to claim 2, wherein the carrier comprises at least one selected from the group consisting of macrogels, succinic acid, urea, pectin, desoxycholic acid, galactomannan, urethane, methylcellulose, hydroxypropylcellulose, polyethylenglycol, poloxamers, polyacrylates, polymethylacrylates, hydroxyalkylxanthine, dextrose, sucrose, polyvinylpyrrolidon, galactose, maltose, xylitol, cyclodextrin, mannitol, sorbitol, HPMC (hydroxypropyl-methylcellulose) and HPMCP (hydroxypropyl methylcellulose phthalate).

9. Process for the preparation of a dispersion of stable amorphous Cinacalcet hydrochloride in a matrix material comprising:
dissolving Cinacalcet hydrochloride in an organic solvent or mixture of organic solvents to obtain a solution of Cinacalcet hycrochloride;
adding a pharmaceutical acceptable carrier to the solution of Cinacalcet hydrochloride; and
removing the solvent to provide a dispersion of stable amorphous Cinacalcet hydrochloride in a matrix material comprising the carrier.

10. Process according to claim 9, wherein the organic solvent solvent comprises at least one selected from the group consisting of acetone, dichloromethane, dioxane, mixtures of dioxane with water or diethylether, dimethylsulfoxyde, ethylacetate, ethylmethylketone, tetrahydrofurane, methanole, 1-proanole, 2-propanole, and 2-propanole in combination with heptane, water or diethylether, or formic acid.

11. Process according to claim 9, wherein the solvent is removed by spray drying, lyophilization or distillation.

12. Process according to claim 9, wherein the carrier comprises at least one selected from the group consisting of macrogels, succinic acid, urea, pectin, desoxycholic acid, galactomannan, urethane, methylcellulose, hydroxypropylcellulose, polyethylenglycol, poloxamers, polyacrylates, polymethylacrylates, hydroxyalkylxanthine, dextrose, sucrose, polyvinylpyrrolidon, galactose, maltose, xylitol, cyclodextrin, mannitol, sorbitol, HPMC (hydroxypropyl-methylcellulose), and HPMCP (hydroxypropyl methylcellulose phthalate).
